(19) United States
(12) Patent Application Publication
(10) Pub. No.: US 2011/0320216 A1
(43) Pub. Date: Dec. 29, 2011

(54) Computer pointing device having medical monitoring features and system for collecting, processing, storing, and dispersing data therefrom

(76) Inventor: Thomas L. Kasmark, Virginia Beach, VA (US)

(21) Appl. No.: 12/803,302

(22) Filed: Jun. 24, 2010

(51) Int. Cl. G06Q 50/00; A61B 5/02; A61B 5/022; G06F 3/033; A61B 5/021

(52) U.S. Cl. 705/2; 345/157; 600/485; 600/499; 600/301

(57) ABSTRACT

A computer pointing device commonly called a mouse that, in addition to functioning as a computer mouse of the prior art, combines a sphygmomanometer and a thermometer. A glucometer or other specialized measurement devices may be attached via a built-in USB or similar interface. Data (e.g., blood glucose readings) from such attached instruments may be processed along with blood pressure, temperature, pulse, etc. A general purpose computer or a dedicated computer-like appliance is connected to the mouse and receives, stores, and processes data relating to measurements made by the mouse or an attached device. Data may be transmitted from the computer to a central data facility where it may be made available to authorized medical personnel and/or institutions.

### FIELD OF THE INVENTION

The invention pertains to devices for collecting vital sign and other data from a subject and, more particularly, to a computer pointing device having built-in and attachable measurement sensors for collecting vital sign data and a system for processing, storing, and dispersing the information collected.

### BACKGROUND OF THE INVENTION

Many people suffer from medical conditions that require periodic monitoring of a health-related parameter related to their bodies. For example, diabetics typically measure blood glucose levels several times throughout the day. Hypertensive people monitor their blood pressure to ensure that it stays within a safe range. Other physical conditions may require periodic monitoring of other physical parameters.

While many devices exist for use by individuals to self-monitor a required parameter, the accuracy of the monitoring may be less than optimum. This may be due to factors such as inaccuracy inherent in inexpensive, “home” type monitoring equipment or, in other cases, lack of skill by the individual in performing the measurements. In addition, record keeping, that is recording the readings obtained from measurements is sometimes inadequate. When record keeping is poor, a physician or other trained licensed medical personnel may not have the necessary historical data to diagnose or evaluate the medical condition of the individual or to prescribe treatment of a medical condition.

In addition, even when an individual keeps good records of accurate measurements, getting that data to more than one medical practitioner becomes problematic. Even as we enter the age of paperless medical records, there is rarely any way for an individual to enter data directly into the system where it may readily be disseminated to multiple practitioners and/or institutions.

### DISCUSSION OF THE RELATED ART

U.S. Pat. No. 7,335,163 for COMBINED COMPUTER MOUSE AND BLOOD PRESSURE SPHYGMOMANOMETER, issued Feb. 26, 2008 to Phillip L. Lam et al. provides a device that combines a well-known computer accessory with a blood pressure measurement system. Measurements made with the LAM et al. mouse may be recorded directly in a computer or other electronic device to which the mouse is electrically connected. The blood pressure data may be stored and averaged or otherwise analyzed by the attached computer using software.

This patent neither teaches nor suggests the novel computer pointing device having medical monitoring features and the attending system of the present invention.

### SUMMARY OF THE INVENTION

In accordance with the present invention there is provided a computer pointing device commonly called a mouse that, in addition to functioning as a computer mouse of the prior art, combines a sphygmomanometer, a thermometer, and at least a glucometer. A built-in USB or similar interface allows other stand-alone measurement devices to be connected to the novel mouse, the measurements made by the devices being processed along with blood pressure and/or blood glucose readings from the novel mouse.

A general purpose computer or a dedicated computer-like appliance is connected to the mouse and receives, stores, and processes data relating to measurements made by the mouse or an attached device. Data may be transmitted from the computer to a central data facility where it may be made available to authorized medical personnel and/or institutions.

It is, therefore, an object of the invention to provide a medical data collection device incorporated into a form such as a computer mouse.

It is another object of the invention to provide a medical data collection device incorporated into a form such as a computer mouse having a data port allowing attachment of external measurement devices.

It is an additional object of the invention to provide a medical data collection device incorporated into a form such as a computer mouse having a data port implemented as a USB port.

It is a further object of the invention to provide a medical data collection device incorporated into a form such as a computer mouse that may be connected to a local controller in the form of a computer or dedicated appliance.

It is a still further object of the invention to provide a medical data collection device incorporated into a form such as a computer mouse wherein medical data collected thereby may be stored and/or analyzed at a local controller.

It is yet another object of the invention to provide a medical data collection device incorporated into a form such as a computer mouse wherein medical data from a local controller may be transmitted to a remote site and may available to authorized medical practitioners or other authorized users of such data.

### BRIEF DESCRIPTION OF THE DRAWINGS

Various objects, features, and attendant advantages of the present invention will become more fully appreciated as the same becomes better understood when considered in conjunction with the accompanying drawings, in which like reference characters designate the same or similar parts throughout the several views, and wherein:

FIG. 1a is a front, perspective, schematic view of a computer pointing device having a sphygmomanometer included within;

FIG. 1b is a detailed perspective view of a tunnel portion of the mouse of FIG. 1a;

FIG. 1c is a front, perspective, schematic view of the computer pointing device of FIG. 1a with exterior cover removed;

FIG. 2 is a rear, perspective, schematic view of the computer pointing device of FIG. 1;

FIG. 3 is a side, perspective, schematic view of a glucometer adapted to be attached to the computer pointing device of FIG. 2;

FIG. 4 is a simplified system block diagram of a local portion of the system of the invention;

FIG. 5 is screen shot of a typical display of data gathered by the computer pointing device of FIG. 1a at a computer system attached thereto; and

FIG. 6 is a simplified, pictorial representation of the data collection, storage, and dispersal system in accordance with the invention.

### DETAILED DESCRIPTION OF THE PREFERRED EMBODIMENT

The present invention provides an apparatus for collecting vital sign information from a human subject incorporated in a computer pointing device such as a computer “mouse”. The mouse is adapted to communicate with a computer or other electronic appliance adapted to receive data representative of a physical parameter collected from the mouse, store the data, optionally perform mathematical manipulation or analysis of the data, and to transmit raw and/or processed data to a remote site.

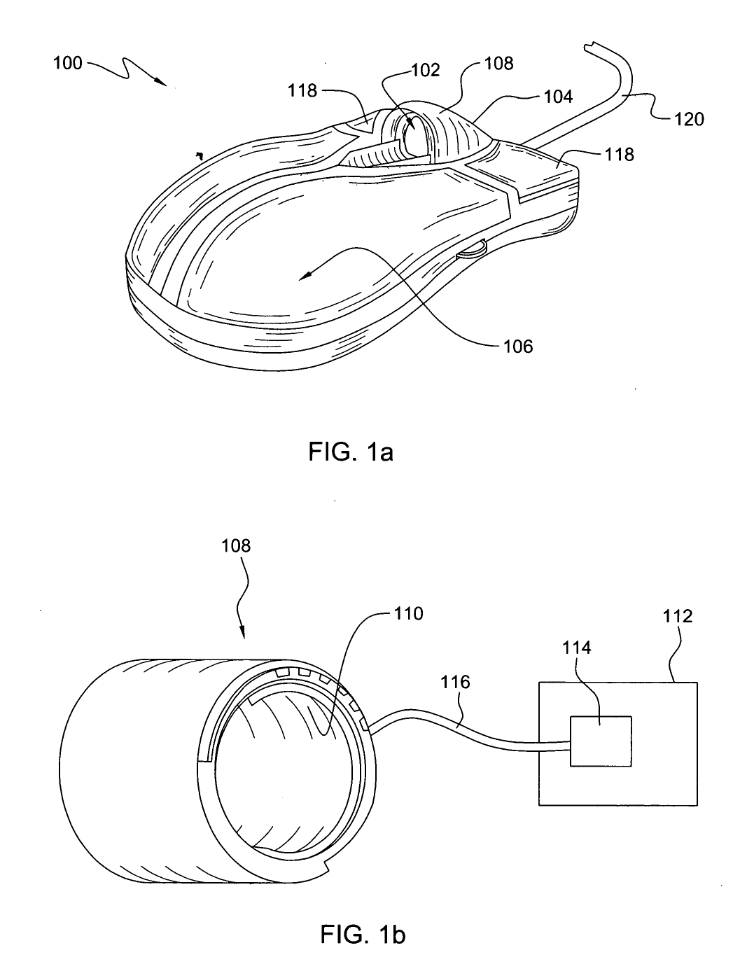

Referring first to FIGS. 1a, 1b, and 1c, there are shown a front, perspective, schematic view of a computer mouse 100 having a built-in sphygmomanometer, not specifically identified, a detailed perspective view of a tunnel 108 portion of the mouse of FIG. 1a, and a front, perspective view of computer mouse 100 but with an outer cover removed, respectively. Mouse 100 resembles a well known mouse of the prior art with the exception of an opening 102 in a housing 104 forming a portion of the upper portion 106 of mouse 100. Opening 102 defines the outer end of a substantially cylindrical tunnel 108 within housing 104 that is sized and configured to circumferentially surround a finger, not shown, of a user of mouse 100.

The inner wall of substantially cylindrical tunnel 108 includes an inflatable structure 110 (e.g., an airbag) that is adapted to selectively expand inwardly to impose pressure on a finger inserted into substantially cylindrical tunnel 108. Inflatable structure 110 is connected to a mechanism 112 that selectively inflates and deflates inflatable structure 110 upon command thereto. This mechanism typically consists of a small electrically-driven compressor or pump 114 connected to inflatable structure by tubing 116 or another suitable conduit, not shown. Such mechanisms are believed to be well known to those of skill in the art and are not further described herein.

Mouse 100 features such as a tracking element, not shown, and one or more switches 118.

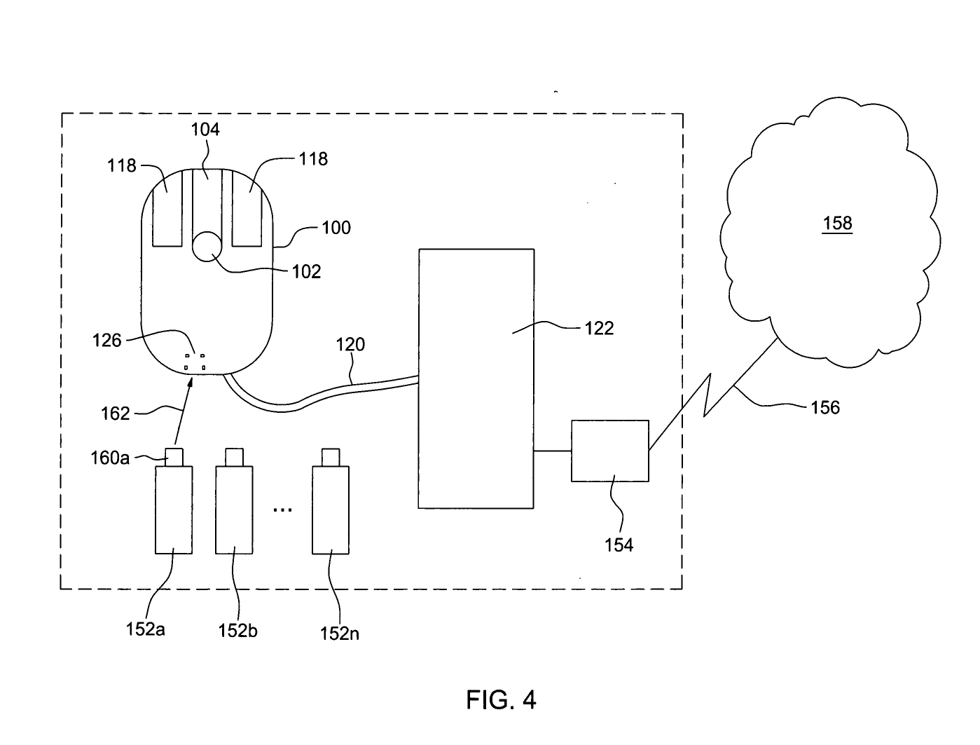

A cable 120 connects mouse 100 to a computer or other appliance 122 (FIG. 4)

It is well known to those of skill in the art to use a sphygmomanometer to also measure pulse rate and such a feature is included in the sphygmomanometer forming a part of mouse 100. In addition, additional sensors, not specifically identified, in or proximate tunnel 108 may be used to read body temperature, pulse oxygen levels, skin resistance, or other measureable body parameters. An EEG subsystem could also be provided. Consequently, the invention is not limited to the particular body parameters chosen for purposes of disclosure but is intended to include any additional body parameters measureable at the finger of a user of mouse 100. Possible additional auxiliary sensors include, but are not limited to serum cholesterol, blood lipids, urine sugar and A1C hemoglobin.

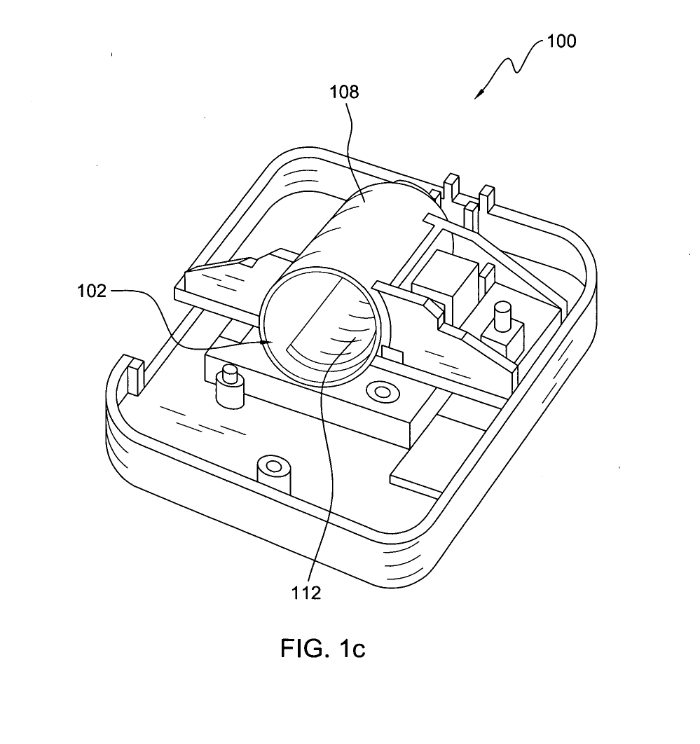

Referring now also to FIG. 2, there is shown a rear, perspective, schematic view of mouse 100. An optional scrolling wheel 124 is disposed on a side of mouse 100. It will be recognized that switches 118 and scrolling wheel 124 form no part on the invention and are included only to illustrate features often found on computer pointing devices such as mouse 100. Consequently, the invention is not changed by the absence or presence of features such as, but not limited to switches 118 and scrolling wheel 124.

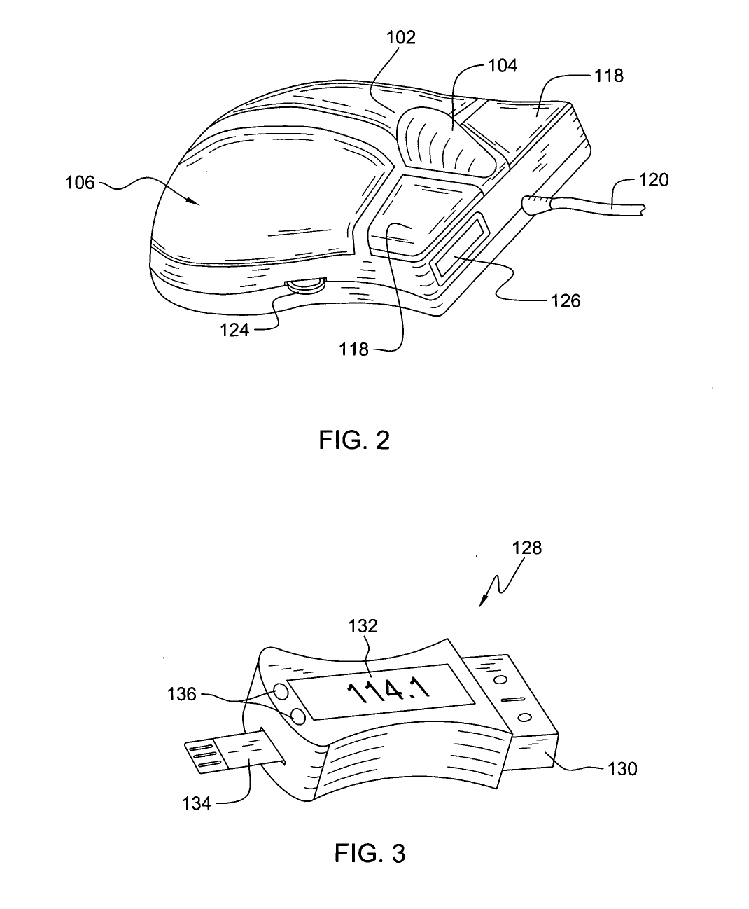

A USB connector 126 is disposed on a rear edge of mouse 100. USB connector 126 is adapted to optionally receive auxiliary measurement devices, for example a glucometer 128 (FIG. 3). As the medical condition of each potential user of the system of the present invention may vary, the inclusion of USB connector 126 allows a user to add measurement devices pertinent to his or her medical condition and consequent need for particular data measurements.

Referring now also to FIG. 3, there is shown a perspective, schematic view of a glucometer 128 having a USB connector 130 adapted to be received in USB connector 126 on mouse 100. Glucometer 128 is similar to stand alone glucometers of the prior art, not shown, and is equipped with a slot to receive a test strip 134. Test strip 134 forms no part of the invention and is included only to show a typical operating configuration of glucometer 128. Glucometer 128 may further optionally contain one or more controls (typically buttons) 136 to performs any necessary setup function, for example but not limited to matching a control number, not shown, of a particular batch, not shown, of test strips 134 to glucometer 128 when required. Glucometer 128 may optionally be provided with a display 132.

While glucometer 128 is shown for purposes of disclosing auxiliary measuring devices attachable to mouse 100 via USB connector 126, it will be recognized that other measurement devices may be contemplated. The invention is not, therefore, considered limited to the auxiliary glucometer 128 chosen for purposes of disclosure. Rather, the invention includes any and all auxiliary instruments designed to measure a human body parameter.

Referring now also to FIG. 4, there is shown a simplified schematic system block diagram of a local portion of the overall system of the present invention, generally at reference number 150. Mouse 100 having a USB connector 126 is shown adjacent a plurality of auxiliary measurement devices 152a, 152b . . . 152n. While devices 152a, 152b, . . . 152n are shown schematically as having a built-in USB connector 160a, 160b . . . 160n adapted for selective interconnection to USB connector 126 on mouse 100, it will be recognized that auxiliary measurement devices 152a, 152b, . . . 152 may be in other form factors, not shown, that may be interconnected to USB port 126 of mouse 100 by a cable, not shown, having a compatible USB connector, not shown, at a distal end thereof. A desired one of auxiliary measurement devices 152a, 152b, . . . 152n is selectively plugged into USB connector 126 of mouse 100 as shown by arrow 162. Glucometer 128 (FIG. 3) is an example of auxiliary measurement devices 152a, 152b . . . 152n.

Cable 120 of mouse 100 is shown connected to computer 122. As used herein in, the term computer is intended to apply to any general purpose computer or dedicated electronic appliance having the capability to interconnect to mouse 100 and an appropriate communications interface 154. Computer 122 is assumed to include all internal and external systems, sub-systems, operating system(s), application software, and peripheral devices, none of which is specifically identified, necessary to perform any necessary control or computational processes. For example, computer 122 may include an additional human interface such as a keyboard and a monitor, neither specifically identified.

A communications interface 154 is provided to communicate data from computer 122 to a remote site. For purposes of disclosure it is assumed that all electronic communication is via a communications link 156 to the Internet, shown schematically at reference number 158.

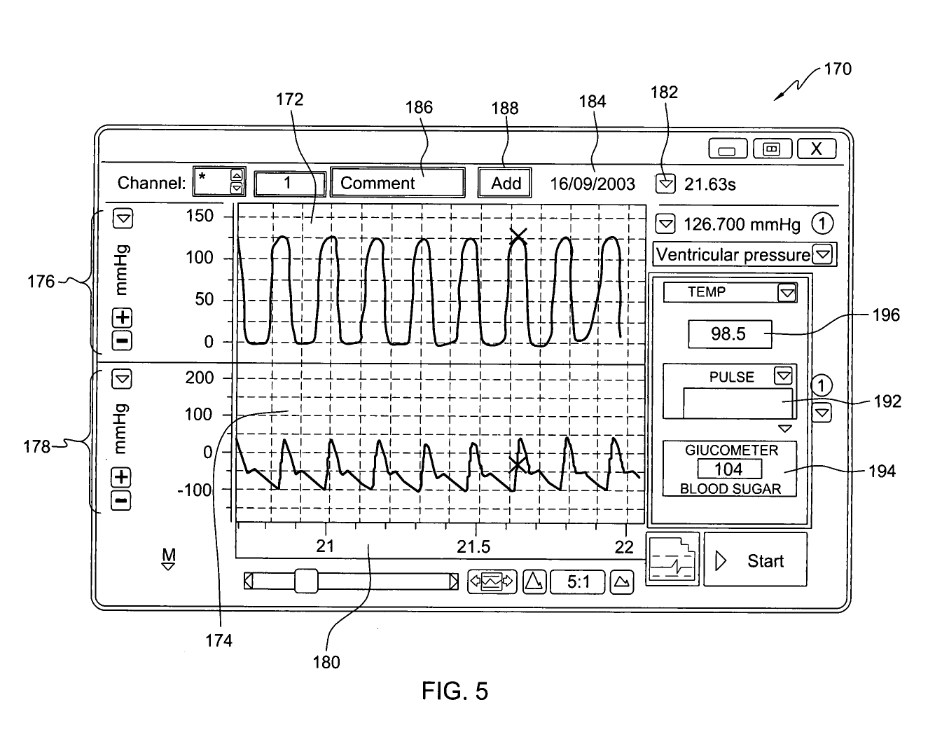

Referring now to FIG. 5, there is shown a typical screen displayed on the monitor, not specifically identified, of computer system 122, generally at reference number 170. A number of data display areas are shown on screen 170. Screen 170 has a number of display areas dedicated to presentation on a single aspect of the data gathered at local system 150, primarily by mouse 100 and ancillary measurement instruments attached thereto by USB connector 126.

For example, display regions 172 and 174 display, respectively a waveform associated with ventricular and arterial blood pressures. Controls 176 and 178, associated with display regions 172, 174 respectively, control the display parameters thereof.

A time scale 180 displays the time (in seconds) during which the waveforms in display regions 174, 174 have been acquired. A control 182 allows selection of a specific time period to be displayed.

An optional comment field 186 is provided for adding descriptive information to the data stored from a particular measurement Control 188 is provided to allow addition of comment 186.

Temperature 190, pulse 192, and blood sugar 194 readings are displayed in respective display regions at the right hand side of screen 170.

Other controls, not specifically identified, believed to be well known to those of skill in the art are provided to control various display parameters and functions.

It will be recognized that numerous display layouts may be conceived and provided to display measurements at local system 150. Consequently, the invention is not considered limited to the specific layout or to the data elements displayed on screen 170 provided for purposes of disclosure. Rather, the invention includes any screen layout and mix of measured values.

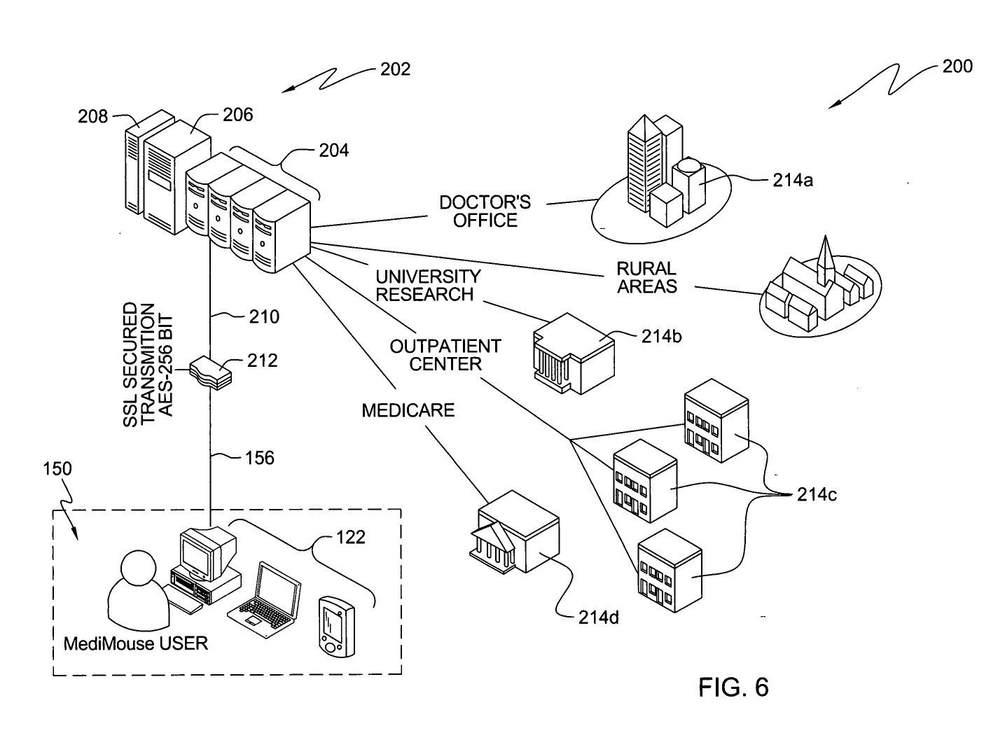

Referring now also to FIG. 6, there is shown a simplified, pictorial diagram of the system for acquiring, storing, transmitting, and dispersing information in accordance with the invention, generally at reference number 200.

A central facility 202 contains processors 204, bulk data storage units 206, and communications controllers 208. Central facility 202 is adapted to receive and store uploaded data from numerous user local systems 150, only one of which is shown, via communications links 156 and 210 from each of the local systems 150.

Secure data communication is maintained on communications links 156, 210 by a security device, shown schematically at reference number 212 and associated with communications links 156 and 210. Data security device 212 may be implemented using a wide range of different protocols and security arrangements believed to be well known to those of skill in the electronic communication arts. For purposes of disclosure, data security device 212 provides a secure sockets layer (SSL) security protocol, specifically and AES-256 bit implementation, to ensure the security of data to and from a local system 150 and the communications controller 208 at central facility 202. SSL is believed to be widely understood by those of skill in the art and, consequently, is not further discussed herein. However, the invention is not considered limited to the SSL method used for purposes of disclosure. Rather, the invention includes the use of either no data security system or any known or yet to be implemented data security system.

Data, not shown, from each local system 150 is transmitted to central data management facility 202 where it is stored (e.g., warehoused) with other data for each identified user. Uploaded data for each individual may be processed, for example, averaged or otherwise statistically analyzed. It is envisioned that either raw data or, in order to conserve storage space, various summaries of the data may be stored.

Authorized Data Consumers (ADCs), typically medical practitioners 214a, research facilities such as university research facility 214b, outpatient clinics 214c, government agencies, for example, Medicare administration 214d, are able to communicate with remote data management facility 202 and, when properly authorized, obtain data for one or more individuals for whom data has been uploaded, stored, summarized and possibly analyzed.

It is anticipated that the system of the invention would be a privately run service to which individuals could subscribe. All or portions of local system 150 could be either purchased outright by a subscriber or leased from the entity providing the service.

Persons wishing to store data would subscribe and obtain necessary portions of local system 150. Each person would be responsible for determining what medical data was to be gathered, with what frequency measurements should be made, time of measurements, etc. Such determinations would presumably be made in consultation with competent, probably licenses, medical professionals.

Subscription fees may be determined by the number, frequency, and/or complexity of tests performed at local system 150. Additional charges could be imposed based upon the amount of data being stored or other similar factors.

ADCs 214a . . . 214n may, upon proper authorization, be allowed free access to the data. In alternate embodiments, ADCs 214a . . . 214n may be charged either a subscription fee or a pay per use charge to obtain information from central data management system 202.

System 200 is advantageous in that individuals may monitor certain medical parameters at home using reliable equipment that is as close to fool proof as possible. Features in either local or remote software may be provided to flag suspicious readings so that they may be repeated if they fail to meet a predetermined criteria. If repeated measurements are suspect, the user may be directed to a medical professional (e.g., doctor's office, clinic, etc.) to have a reading verified.

The ability to routinely monitor health related values from home alleviates the need for frequent trips to a doctor's office, emergency room, clinic, etc., thereby freeing those resources for more critical care related utilization. Further, it saves significant amounts of time for the user who avoids the time overhead and expense of transportation (public or private).

Since other modifications and changes varied to fit particular operating requirements and environments will be apparent to those skilled in the art, the invention is not considered limited to the example chosen for purposes of disclosure, and covers all changes and modifications which do not constitute departures from the true spirit and scope of this invention.

Having thus described the invention, what is desired to be protected by Letters Patent is presented in the subsequently appended claims.

What is claimed is:

1. A computer pointing device adapted to obtain measurements of a physical parameter of a user thereof, comprising:
a) a computer pointing device having a body portion adapted to be grasped by a hand of a user thereof and moved thereby in at least two-dimensional space, said computer pointing device providing an electrical signal representative of a position thereof in two-dimensional space, said computer pointing device further comprising at least one switch selectively movable between at least two states and adapted to provide an electrical signal representative of a selected state thereof;
b) an opening disposed on said housing to receive at least a finger of said hand of said user;
c) means for measuring at least one physical parameter of said user disposed adjacent said opening, said means for measuring providing at least one electrical signal representative of said physical parameter being measured; and
d) a data port disposed on said body portion for providing an electrical connection adapted to receive an electrical signal from an ancillary measurement device electrically connected thereto.

2. The computer pointing device adapted to obtain measurements of a physical parameter of a user thereof as recited in claim 1, wherein said means for measuring at least one physical parameter comprises means for measuring blood pressure.

3. The computer pointing device adapted to obtain measurements of a physical parameter of a user thereof as recited in claim 2, wherein said means for measuring at least one physical parameter comprises means for measuring blood pressure comprising:
i) a tunnel disposed in said body portion adjacent to and contiguous with said opening; and
ii) a sphygmomanometer disposed adjacent said tunnel and adapted for operative interaction with a finger of a hand of said user when inserted into said tunnel via said opening.

4. The computer pointing device adapted to obtain measurements of a physical parameter of a user thereof as recited in claim 3, wherein said sphygmomanometer comprises:
i) an inflatable structure lining at least a portion of said tunnel and adapted to at least partially surround a finger of a hand of said user when inserted into said tunnel via said opening;
ii) means for selectively inflating and deflating said inflatable structure operatively connected thereto; and
iii) at least one sensor operatively connected to at least one of said inflatable structure, and said tunnel, said at least one sensor generating an electrical signal representative of at least said blood pressure.

5. The computer pointing device adapted to obtain measurements of a physical parameter of a user thereof as recited in claim 3, wherein said means for measuring further comprises means for measuring at least one of the parameters selected from the group: body temperature, a pulse oxygen level, skin resistance, and another measureable body parameter.

6. The computer pointing device adapted to obtain measurements of a physical parameter of a user thereof as recited in claim 3, wherein said tunnel is disposed on an upper surface of said body portion.

7. The computer pointing device adapted to obtain measurements of a physical parameter of a user thereof as recited in claim 1, further comprising:
e) a scrolling control disposed on said body portion and operable by said hand of said user, said scrolling control providing an electrical signal representative of a position thereof.

8. The computer pointing device adapted to obtain measurements of a physical parameter of a user thereof as recited in claim 1, wherein said data port comprises a USB connector.

9. The computer pointing device adapted to obtain measurements of a physical parameter of a user thereof as recited in claim 8,
e) an ancillary measurement device electrically connected to said USB connector.

10. The computer pointing device adapted to obtain measurements of a physical parameter of a user thereof as recited in claim 8, wherein said ancillary measurement device comprises at least one selected from the group: a glucometer, a serum cholesterol measurement device, a serum lipids measurement device, a urine sugar measurement device, and an A1C hemoglobin measurement device.

11. A system for obtaining a measurement of a physical parameter of a human body, comprising:
a) a computer pointing device having a body portion adapted to be grasped by a hand of a user thereof and moved thereby in at least two-dimensional space, said computer pointing device providing an electrical signal representative of a position thereof in two-dimensional space, said computer pointing device further comprising at least one switch selectively movable between at least two states and adapted to provide an electrical signal representative of a selected state thereof, an opening disposed on said housing to receive at least a finger of said hand of said user, means for measuring at least one physical parameter of said user disposed adjacent said opening, said means for measuring providing at least one electrical signal representative of said physical parameter being measured and a data port disposed on said body portion for providing an electrical connection adapted to receive an electrical signal from an ancillary measurement device electrically connected thereto;
b) a controller operatively connected to said computer pointing device and adapted to receive said electrical signals therefrom, said controller comprising means for processing said electrical signals and computing a value representative of said at least one physical parameter;
c) means from communicating said value representative of said at least one physical parameter to an apparatus remote from said controller.

12. The system for obtaining a measurement of a physical parameter of a human body as recited in claim 10, wherein said controller further comprises a display for graphically displaying said value representative of said at least one physical parameter.

13. The system for obtaining a measurement of a physical parameter of a human body as recited in claim 10, wherein said means for communicating comprises an Internet connection.

14. The system for obtaining a measurement of a physical parameter of a human body as recited in claim 10, wherein said means for communicating comprises a data encryption device.

15. A system for collecting, storing, and distributing medical data, comprising:
a) a plurality of medical data collection systems, each comprising a computer pointing device having a body portion adapted to be grasped by a hand of a user thereof and moved thereby in at least two-dimensional space', said computer pointing device providing an electrical signal representative of a position thereof device in two-dimensional space, said computer pointing device further comprising at least one switch selectively movable between at least two states and adapted to provide an electrical signal representative of a selected state thereof, an opening disposed on said housing to receive at least a finger of said hand of said user, means for measuring at least one physical parameter of said user disposed adjacent said opening, said means for measuring providing at least one electrical signal representative of said physical parameter being measured and a data port disposed on said body portion for providing an electrical connection adapted to receive an electrical signal from an ancillary measurement device electrically connected thereto, a controller operatively connected to said computer pointing device and adapted to receive said electrical signals therefrom, said controller comprising means for processing said electrical signals and computing a value representative of said at least one physical parameter, and means for communicating said value representative of said at least one physical parameter to a an apparatus remote from said controller;
b) a central data collection facility located remotely from at least one of said plurality of medical data collection facilities and operatively connected to said means for communicating of said at least one of said plurality of medical data collection facilities and adapted to receive medical data therefrom;
c) means for analyzing medical data disposed within said central data collection facility for analyzing raw medical data from said at least one of said medical data collection facilities and yielding analyzed medical data;
d) means for storing at least one selected from the group: said raw medical data, and said analyzed medical data disposed within said central data collection facility;
e) means for selectively distributing at least one selected from the group: said raw medical data, and said analyzed medical data to authorized medical data consumers.

16. The system for collecting, storing, distributing medical data as recited in claim 15, wherein said means for selectively distributing at least one selected from the group: said raw medical data, and said analyzed medical data to authorized data consumers comprises a security sub-system for preventing unauthorized access to said at least one selected from the group: said raw medical data, and said analyzed medical data.

17. The system for collecting, storing, and distributing medical data as recited in claim 15, wherein said security sub-system comprises a password.
